(19) United States
(12) Patent Application Publication
(10) Pub. No.: US 2025/0057417 A1
(43) Pub. Date: Feb. 20, 2025

(54) Ophthalmic Microscope System and System, Method and Computer Program for an Ophthalmic Microscope System

(71) Applicant: LEICA INSTRUMENTS (SINGAPORE) (84) PTE. LTD., Singapore (SG)

(72) Inventor: Gao YANG, Singapore (SG)

(21) Appl. No.: 18/728,119

(22) PCT Filed: Jan. 5, 2023
(86) PCT No.: PCT/EP2023/050174

§ 371 Date: Jul. 11, 2024

(30) Foreign Application Priority Data
Jan. 11, 2022 (DE) 10 2022 100 503.8

(51) Int. Cl. A61B 3/13; A61B 3/14; G16H 40/63

(52) U.S. Cl. CPC A61B 3/13; A61B 3/14; G16H 40/63

(57) ABSTRACT

Examples relate to a system (110), method, and computer program for an ophthalmic microscope system, and to a corresponding ophthalmic microscope system. The system is configured to obtain imaging sensor data from an optical imaging sensor (122) of a microscope of the ophthalmic microscope system. The system is configured to determine information on an anatomical feature of an eye shown in the imaging sensor data. The system is configured to control an illumination system (130) of the ophthalmic microscope system to adjust a property of a light beam used for red reflex illumination of the eye based on the information on the anatomical feature.

### TECHNICAL FIELD

Examples relate to a system, method, and computer program for an ophthalmic microscope system, and to a corresponding ophthalmic microscope system.

### BACKGROUND

In ophthalmic surgical microscopes, a main light is used to illuminate the anterior surface of the eye, and a red reflex light is used to illuminate the retina to obtain a bright orange reflection, to make the transparent structures, such as the lens capsules, visible upon deformation.

Ideally, the red reflex light should provide clear and uniform reflection from the retina without affecting the illumination on the eye's front surface.

In many ophthalmic microscopes, the red reflex light covers a static circular area centered around the middle of the field of view. When the radius is set to be small, the red reflex may be dim or invisible when the eye shifts around. On the other hand, when the radius is set to be large, light is spilled over to the anterior of the eye, which may render the surrounding area overly bright. The effect of light spilling over is especially prominent in digital heads-up surgery where digital video cameras have a narrower dynamic range than human observers.

Low red reflex intensity often results in poor visibility. High red reflex intensity may cause unnecessary damage to the retina. Each eye may have a different optimal red reflex intensity setting and may require manual adjustment during surgery. Certain procedures such as hydrodissection can also significantly alter the light intensity needed to achieve good visualization.

In some ophthalmic microscopes, the red reflex light intensity can be manually adjusted independent of the main light, and its coverage can be adjusted using an iris diaphragm via a fly-by-wire knob. However, this is a manual process, not an automatic process for following the eye.

There may be a desire for an improved concept for an ophthalmic surgical microscope.

### SUMMARY

This desire is addressed by the subject-matter of the independent claims.

The concept proposed in the present disclosure is based on the finding that the position of the eye during ophthalmic surgery often is not entirely static, i.e., the eye may move (or be moved) during surgery. Moreover, the red reflex effect may change during surgery, e.g., as a result of hydrodissection. Therefore, a static red reflex light setting may be insufficient to deal with such changes. In the proposed concept, image analysis is used to determine information of an anatomical feature of the eye, such as the position of the pupil or the limbus, or the intensity of the red reflex effect, with the information being used to adjust the light beam being used for red reflex illumination, e.g., by adjusting the path of the light beam, and thus target area on the eye, or by adjusting the intensity of the light beam. Accordingly, the center and radius of the red reflex illumination iris can be automatically controlled based on live surgical video. Moreover, the intensity of the red reflex illumination can be automatically adjusted based on the live surgical video. Various examples of the present disclosure may provide means for automatic red reflex light pattern and/or intensity control.

Various examples of the present disclosure relate to a system for an ophthalmic microscope system. The system is used to control various aspects of the ophthalmic microscope system and may be integrated in the ophthalmic microscope system. The system comprises one or more processors and one or more storage devices. The system is configured to obtain imaging sensor data from an optical imaging sensor of a microscope of the ophthalmic microscope system. The system is configured to determine information on an anatomical feature of an eye shown in the imaging sensor data. The system is configured to control an illumination system of the ophthalmic microscope system to adjust a property of a light beam used for red reflex illumination of the eye based on the information on the anatomical feature. By determining the information on the anatomical feature, the system can determine whether the light beam is to be adjusted after a movement or other change that would affect the red reflex and can accordingly adjust the property of the light beam.

In some examples, the anatomical feature is a pupil of the eye. The system may be configured to determine information on the pupil of the eye, and to control the illumination system to adjust the property of the light beam based on the information on the pupil of the eye. Alternatively, the anatomical feature may be (or relate to) a limbus of the eye. The system may be configured to determine information on the limbus of the eye, and to control the illumination system to adjust the property of the light beam based on the information on the limbus of the eye. Both the position of the pupil and of the limbus may be used to determine a direction and/or a diameter of the light beam.

With both the pupil and the limbus, the position of the respective feature (and, optionally, its size) is relevant with respect to the light beam. For example, the system may be configured to determine a position of the anatomical feature of the eye, such as the pupil or limbus. The system may be configured to control the illumination system to adjust a path of the light beam based on the position of the anatomical feature of the eye. In other words, the target of the light beam may be adjusted according to the position of the anatomical feature of the eye.

There are various means that can be used to adjust the path of the light beam. For example, the system may be configured to control one or more motors to adjust a position of at least one of a light source and an iris of the illumination system in order to adjust the path of the light beam. By moving the iris and/or the light source, the path of the light beam may be shifted perpendicular to the direction of the light beam.

Alternatively (or additionally), the system may be configured to control a display device being configured to modulate the light beam in order to adjust the path of the light beam. For example, the light beam may pass through a portion (e.g., a first set of pixels) of the display device, with the position and size of the portion determining the path (and width) of the light beam. Alternatively, the light beam may be reflected by a portion (e.g., a first set of pixels) of the display device, with the position and size of the portion determining the reflected path (and width) of the light beam.

As outlined above, the size of the portion of the display device affects the width (e.g., diameter) of the light beam. Alternatively, the above-mentioned iris may be adjusted to adjust the width of the light beam. The system may be configured to determine a size of the anatomical feature of the eye, and to control the illumination system to adjust a width of the light beam based on the size of the anatomical feature of the eye. For example, the system may be configured to control an iris of the illumination system in order to adjust the width (or diameter) of the light beam. Alternatively, the system may be configured to control a display device being configured to modulate the light beam in order to adjust the width of the light beam. The width of the light beam may be adjusted to account for different sizes of pupils or to account for changes in the size (or position) of the pupil.

During ophthalmic surgery, the red reflex may change due to surgical operations being performed by the surgeon, such as hydrodissection. Therefore, the proposed concept may be used to compensate for such changes. The system may be configured to determine a brightness or contrast of the red reflex illumination of the eye. The system may be configured to control the illumination system to adjust an intensity of the light beam based on the brightness or contrast of the red reflex illumination. For example, the system may be configured to control a light source of the illumination system in order to adjust the intensity of the light beam. By adjusting the intensity of the light beam, the visibility of the red reflex may be kept at a suitable level.

In general, the proposed concept may be used during surgery, to adjust the light beam to changes that occur during surgery. In other words, the proposed system may react to changes in the anatomical feature and adjust the light beam accordingly after a change (e.g., in response to a change). For example, the system may be configured to update the information on the anatomical feature over a sequence of frames of the imaging sensor data, and to control the illumination system to adjust the property of the light beam upon detection of change in the information on the anatomical feature of the eye.

In various examples, machine learning (i.e., “artificial intelligence”) may be employed to determine the information on the anatomical feature. For example, the system may be configured to detect the anatomical feature within the imaging sensor data using a machine-learning model being trained to detect the anatomical feature in imaging sensor data. The system may be configured to determine the information on the anatomical feature based on an output of the machine-learning model. For example, machine-learning-based object detection may be used to track the anatomical feature and/or to determine the extent (e.g., position and size) of the anatomical feature, and to use said information to adjust the property of the light beam. Accordingly, the output of the machine-learning model may comprise at least one of information on a position and information on an outline of the anatomical feature. For example, the machine-learning model may be one of a convolutional neural network-based object detector and a gradient-based object detector. Both types of detectors are suitable for determining the position and/or outline of the anatomical feature.

In some examples, the illumination may be modulated for the purpose of determining the information on the anatomical feature. For example, the coverage of the light beam may be temporarily increased (e.g., set to maximum), and the resulting difference between the red reflex and adjacent regions may be used to determine the position of the pupil. For example, the system may be configured to control the illumination system to temporarily increase a beam width of the light beam, and to determine the information on the anatomical feature based on imaging sensor data generated based on the increased beam width. Alternatively, or additionally, the system may be configured to control the illumination system to temporarily disable the light beam. The system may be configured to compare imaging sensor data generated while the light beam is disabled with imaging sensor data generated while the light beam is enabled. The system may be configured to determine the information on the anatomical feature based on the comparison. For example, the pupil may be determined from areas that are brightened significantly when the light beam is enabled.

Various examples of the present disclosure relate to an ophthalmic (surgical) microscope system comprising the above system, the microscope, and the illumination system.

As outlined above, different means may be used to adjust the path of the light beam. For example, the position of a light source and/or of an iris may be moved to adjust the path of the light beam. For example, the illumination system may comprise one or more motors for adjusting the position of at least one of a light source and an iris of the illumination system. The system may be configured to control the one or more motors to adjust the position of at least one of a light source and an iris of the illumination system in order to adjust a path of a light beam being emitted by the illumination system.

Alternatively, the light beam may be transmitted through, or reflected by, a display device. For example, the illumination system may comprise a display device for modulating a light beam being emitted by the illumination system. The system may be configured to control the display device in order to adjust at least one of a path and a width of the light beam.

In some examples, the light beam may be reflected off a portion of the display device. For example, the illumination system may comprise a light source being configured to emit the light beam and the display device. The display device may be configured to selectively reflect the light beam towards the eye, thereby modulating the light beam.

Alternatively, the light beam may be transmitted through the display device. Again, the illumination system may comprise a light source being configured to emit the light beam and the display device. The display device may be configured to modulate the light beam as the light beam passes through the display device towards the eye.

Various examples of the present disclosure relate to a corresponding method for an ophthalmic microscope system. The method comprises obtaining imaging sensor data from an optical imaging sensor of a microscope of the ophthalmic microscope system. The method comprises determining information on an anatomical feature of an eye shown in the imaging sensor data. The method comprises controlling an illumination system of the ophthalmic microscope system to adjust a property of a light beam used for red reflex illumination of the eye based on the information on the anatomical feature.

Various examples of the present disclosure relate to a corresponding computer program with a program code for performing the above method when the computer program is executed on a processor.

### SHORT DESCRIPTION OF THE FIGURES

Some examples of apparatuses and/or methods will be described in the following by way of example only, and with reference to the accompanying figures, in which

FIG. 1a shows a block diagram of an example of a system for an ophthalmic microscope system;

FIG. 1b shows a schematic diagram of an example of an ophthalmic microscope system;

FIGS. 1c to 1e shows schematic diagrams of examples of an ophthalmic microscope system, and in particular of different illumination systems of the ophthalmic microscope system.

FIG. 2 shows a flow chart of an example of a method for an ophthalmic microscope system;

FIG. 3 shows a block diagram of an example of components of the proposed ophthalmic microscope system;

FIG. 4 shows a schematic diagram of an example of a mechanically adjustable iris;

FIG. 5 shows a schematic diagram of an example of an electronically adjustable iris; and

FIG. 6 shows a schematic diagram of a system comprising a microscope and a computer system.

### DETAILED DESCRIPTION

Various examples will now be described more fully with reference to the accompanying drawings in which some examples are illustrated. In the figures, the thicknesses of lines, layers and/or regions may be exaggerated for clarity.

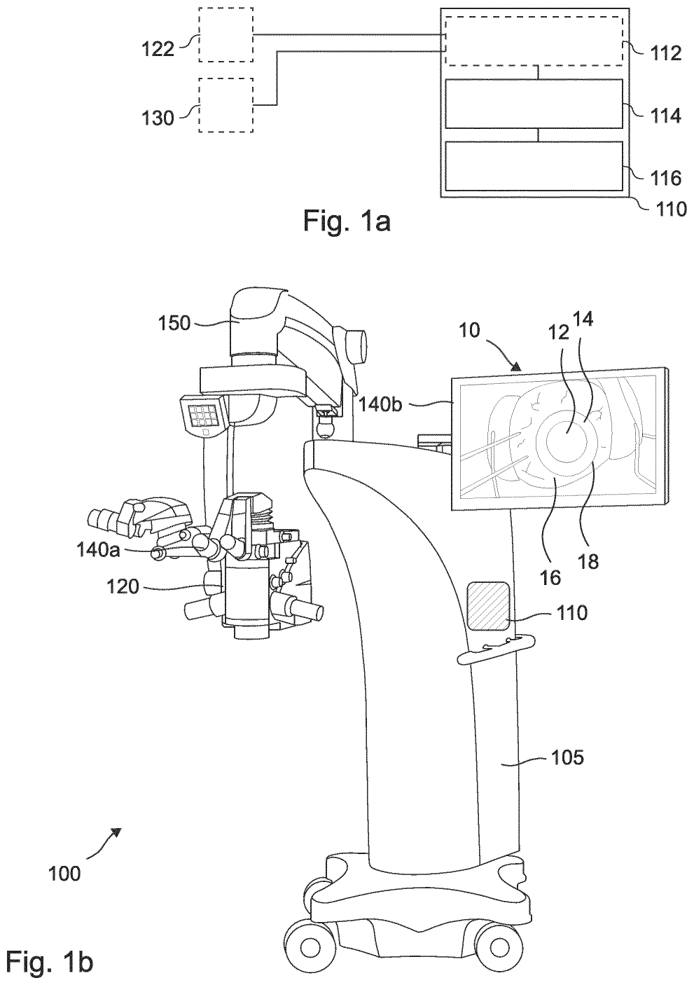

FIG. 1a shows a block diagram of an example of a system 110 for an ophthalmic microscope system 100. The system 110 is tasked with controlling various aspects of a microscope 120 of the ophthalmic microscope system and of the entire ophthalmic microscope system and/or with processing various types of sensor data of the ophthalmic microscope system. Consequently, the system 110 may be implemented as a computer system, which interfaces with the various components of the ophthalmic microscope system.

The system 110 comprises, as shown in FIG. 1a, one or more processors 114 and one or more storage devices 116. Optionally, the system further comprises one or more interfaces 112. The one or more processors 114 are coupled to the one or more storage devices 116 and to the optional one or more interfaces 112. In general, the functionality of the system is provided by the one or more processors 114, in conjunction with the one or more interfaces 112 (for exchanging information, e.g., with at least one optical imaging sensor 122; 124 of the microscope 120, with an illumination system 130 of the ophthalmic microscope system, and/or with a display device 140a; 140b (as shown in FIG. 1b) of the surgical microscope system) and/or with the one or more storage devices 116 (for storing and/or retrieving information). The system is configured to obtain imaging sensor data from the at least one optical imaging sensor 122; 124 of the microscope 120 of the ophthalmic microscope system. The system is configured to determine information on an anatomical feature of an eye shown in the imaging sensor data. The system is configured to control the illumination system 130 of the ophthalmic microscope system to adjust a property of a light beam used for red reflex illumination of the eye based on the information on the anatomical feature.

In the proposed concept, three components of the ophthalmic microscope system 100 interact—the system 110, which processes the imaging sensor data of the microscope and controls the illumination system, the microscope 120, which is used to generate the imaging sensor data, and the illumination system 130, which is used to provide the red reflex illumination.

FIG. 1b shows a schematic diagram of an example of an ophthalmic microscope system 100 comprising the system 110, the microscope 120, and the illumination system (not shown in FIG. 1b). In general, a (ophthalmic) microscope system is a system that comprises a microscope 120 and additional components, which are operated together with the microscope. In other words, a microscope system is a system that comprises the microscope and one or more additional components, such as the system 110 (which is a computer system being adapted to control and, for example, process imaging sensor data of the microscope), the illumination system 130 (which is used to illuminate an object being imaged by the microscope), additional sensors, displays etc.

The ophthalmic microscope system (or ophthalmic surgical microscope system) 100 shown in FIG. 1b comprises a number of optional components, such as a base unit 105 (comprising the system 110) with a (rolling) stand, ocular displays 140a that are arranged at the microscope 120, an auxiliary display 140b that is arranged at the base unit and a (robotic or manual) arm 150 which holds the microscope 120 in place, and which is coupled to the base unit 105 and to the microscope 120. In general, these optional and non-optional components may be coupled to the system 110, which may be configured to control and/or interact with the respective components.

The proposed concept is based on analyzing the imaging sensor data of the at least one optical imaging sensor 122; 124 of the microscope 120 of the ophthalmic microscope system. In general, a microscope, such as the microscope 120, is an optical instrument that is suitable for examining objects that are too small to be examined by the human eye (alone). For example, a microscope may provide an optical magnification of a sample, such as an eye 10 shown in FIGS. 1b to 1e. In modern microscopes, the optical magnification is often provided for a camera or an imaging sensor, such as the at least one optical imaging sensor 122; 124 of the microscope 120. In particular, the microscope 120 may be a stereoscopic microscope, and the optical magnification may be provided for two separate optical imaging sensors 122; 124, as shown in FIGS. 1c to 1e. The microscope 120 may further comprise one or more optical magnification components that are used to magnify a view on the sample, such as an objective (i.e., lens).

There are a variety of different types of microscopes. If the microscope is used in the medical or biological fields, the object being viewed through the microscope may be a sample of organic tissue, e.g., arranged within a petri dish or present in a part of a body of a patient. In the present disclosure, the microscope 120 is a microscope of an ophthalmic microscope system, i.e., a microscope that is to be used during an ophthalmic surgical procedure, i.e., during eye surgery. Accordingly, the object being viewed through the microscope, and shown in the image data, may be an eye 10 of the patient being operated during the surgical procedure.

The microscope 120 comprises the at least one optical imaging sensor 122; 124, which is configured to provide the imaging sensor data being processed by the system 100. In other words, the system 110 is configured to perform image processing on the imaging sensor data, to determine the information on the anatomical feature of an eye shown in the imaging sensor data. In the present concept, there are at least three aspects of the anatomical feature that are of interest—the position of the anatomical feature, the extent (i.e., size) of the anatomical feature, and the effect of the light beam on the anatomical feature (i.e., the intensity of the red reflex). Accordingly, the information on the anatomical feature may comprise at least one of information on a position of the anatomical feature, information on an outline of the anatomical feature (e.g., information on a width/diameter of the anatomical feature), and information on an intensity of the red reflex observed through the anatomical feature. The system may be configured to determine at least one of the information on the position of the anatomical feature, the information on the outline of the anatomical feature, and the information on the intensity of the red reflex observed through the anatomical feature by processing the imaging sensor data.

In ophthalmology, the red reflex is caused by the light beam of the red reflex illumination illuminating the back of the eye (the fundus) through the pupil, causing a bright orange reflection that shows the pupil to be bright orange in the imaging sensor data. To obtain the red reflex, the light beam of the red reflex illumination is to be transmitted through the pupil, to avoid light spilling over to the anterior of the eye, rendering the area surrounding the pupil overly bright. In effect, the position (and outline) of the pupil is relevant for the purpose of targeting the light beam. Therefore, the anatomical feature may be or comprise the pupil of the eye, with the system being configured to determine information on the pupil of the eye. However, in some cases, the transition between the pupil and the iris may be hard to distinguish. Therefore, instead of the pupil (or in addition to the pupil), the limbus of the eye (i.e., the transition between the cornea and the sclera) may be used as reference. Accordingly, the anatomical feature may be or comprise the limbus of the eye, with the system being configured to determine information on the limbus of the eye. In FIGS. 1b and 1c, the relevant components of the eye are illustrated, with FIG. 1b showing, in the auxiliary display 140b, the eye 10 with the pupil 12 and the iris 14 (both covered by the cornea), the sclera 16, and the limbus 18 between the sclera 16 and the cornea. In FIG. 1c, the eye 10 is shown in a cross section, showing the sclera 16, the cornea 17, and the limbus 18 between the sclera 16 and the cornea 17. Since the sclera appears (mostly) bright and the cornea appears darker (due to showing the iris) in the imaging sensor data, the limbus may be clearly distinguishable at the transition between bright and dark.

To detect and track the pupil (red reflex boundary) or the limbus, several approaches can be considered. In some examples, machine-learning may be used to detect the pupil or limbus in the imaging sensor data. For example, the system may be configured to detect the anatomical feature within the imaging sensor data using a machine-learning model being trained to detect the anatomical feature in imaging sensor data, and to determine the information on the anatomical feature based on an output of the machine-learning model.

Some examples are thus based on machine learning. Machine learning may refer to algorithms and statistical models that computer systems may use to perform a specific task without using explicit instructions, instead relying on models and inference. For example, in machine-learning, instead of a rule-based transformation of data, a transformation of data may be used, that is inferred from an analysis of historical and/or training data. For example, the content of images may be analyzed using a machine-learning model or using a machine-learning algorithm. In order for the machine-learning model to analyze the content of an image, the machine-learning model may be trained using training images as input and training content information as output. By training the machine-learning model with a large number of training images and/or training sequences (e.g. words or sentences) and associated training content information (e.g. labels or annotations), the machine-learning model “learns” to recognize the content of the images, so the content of images that are not included in the training data can be recognized using the machine-learning model. The same principle may be used for other kinds of sensor data as well: By training a machine-learning model using training sensor data and a desired output, the machine-learning model “learns” a transformation between the sensor data and the output, which can be used to provide an output based on non-training sensor data provided to the machine-learning model. The provided data (e.g. sensor data, meta data and/or image data) may be preprocessed to obtain a feature vector, which is used as input to the machine-learning model.

In the present context, the machine-learning model is trained to detect the anatomical feature within the imaging sensor data. In this context, the term “detect” might not only refer to the mere presence of the anatomical feature in the imaging sensor data, but also to the position and/or extent of the anatomical feature in the imaging sensor data. In object detection, which the machine-learning model is trained to perform, the output of the machine-learning model usually is a so-called bounding box, which is a (usually) rectangular box defined by coordinates, with the bounding box encompassing the feature when the bounding box is overlaid over the imaging sensor data. Such a bounding box is defined by the position of the vertices of the bounding box, thus providing the position of the anatomical feature, and also an outline (e.g., a rectangular outline) of/around the anatomical feature. Accordingly, the output of the machine-learning model may comprise at least one of information on a position and information on an outline of the anatomical feature. In this context, the information on the outline may be a set of coordinates defining the rectangular bounding box around the anatomical feature, or one or more coordinates and information on a shape of the outline defined a non-rectangular outline more closely tracking the boundaries of the anatomical feature.

In some examples, the machine-learning model may be a convolutional neural network-based object detector. For example, a convolution neural network-based object detector may be used that is trained on labelled eye images. Using the convolution neural network-based object detector, the pupil area (or the limbus) can be identified from the live surgical video. Examples of such detectors include SSD (Single Shot Detector), YOLO (You Only Look Once), and Faster RCNN (Region Based Convolutional Neural Networks). To train such a detector, a plurality of sample images of eyes and a plurality of sets of desired output (e.g., coordinates of the bounding box, coordinates, and information on a shape of the outline, i.e., the “label” of the labelled eye images) may be used with a supervised learning-based approach.

Alternatively, the machine-learning model may be a gradient-based object detector. In other words, the pupil area (or the limbus) can also be identified using a gradient-based object detector such as Haar Cascade or HOG (Histogram of Oriented Gradients). Such machine-learning models operate by detecting edges within the imaging sensor data, e.g., by first transforming the imaging sensor data into a representation that is based on a derivative of the respective pixels (e.g., relative to adjacent pixels). Again, a supervised learning approach based on the labelled eye images may be used to detect the anatomical feature of interest.

Alternatively, the pupil area can be initially located by increasing the red reflex coverage, e.g., by setting the red reflex coverage to its maximum, and segmenting the orange-coloured areas that are likely to correspond with red reflex. In other words, the system may be configured to control the illumination system to temporarily increase a beam width of the light beam (e.g., to a maximal supported beam width), and to determine the information on the anatomical feature based on imaging sensor data generated based on the increased beam width. For example, the imaging sensor data may show one or more areas that are shown in orange may correspond to the red reflex. The system may be configured to segment the imaging sensor data (e.g., using a machine-learning model being trained for image segmentation) to determine the one or more areas shown in orange. This may give several candidate regions, which can be filtered based on size and shape priors to isolate the pupil area. The system may thus be configured to filter the one or more areas based on their shape and/or size to isolate one area as pupil. Subsequently, corner features may be extracted from just outside of the pupil and their locations tracked as a proxy to the pupil.

The red reflex light can also be toggled, and the before and after images compared to identify the areas that has brightened significantly. In other words, the system may be configured to control the illumination system to temporarily disable the light beam, to compare imaging sensor data generated while the light beam is disabled with imaging sensor data generated while the light beam is enabled, and to determine the information on the anatomical feature based on the comparison. For example, the system may be configured to determine one or more areas within the imaging sensor data that appear substantially (e.g., at least 10%, or at least 25%, or at least 50%) brighter in the imaging sensor data generated while the light beam is enabled compared with the imaging sensor data generated while the light beam is disabled. The pupil can then be identified from these areas based on size, shape, and other image statistics such as brightness. Accordingly, the system may be configured to detect the pupil among the one or more areas based on a size, a shape and/or a brightness of the one or more areas.

Once the information on the anatomical feature is determined, the system controls the illumination system 130 of the ophthalmic microscope system to adjust the at least one property of the light beam. As outline above, there are (at least) three properties of the light beam that are of interest with respect to red reflex illumination—the path of the light beam, the width of the light beam, and the intensity of the light beam. These properties can be derived from the information on the anatomical feature, in particular from the information on the position of the anatomical feature, the information on the outline of the anatomical feature, and the information on the intensity of the red reflex observed through the anatomical feature (i.e., pupil). For example, the system may be configured to determine the (desired) path of the light beam based on the information on the position of the anatomical feature, e.g., by selecting a path that intersects with the position of the anatomical feature. The system may be configured to determine the (desired) width of the light beam based on the information on the outline of the anatomical feature, by selecting a width that fits into the outline and/or fills the outline of the anatomical feature (as far as possible). Finally, as will be introduced in more detail at a later stage, the system may be configured to determine the (desired) intensity of the light beam based on the information on the intensity of the red reflex observed through the pupil.

In the following, the (desired) path and the (desired) width of the light beam are discussed. In particular, the system may be configured to determine a position of the anatomical feature of the eye (i.e., the information on the position of the anatomical feature), and to control the illumination system to adjust a path of the light beam based on the position of the anatomical feature of the eye, e.g., such that the beam of light intersects with the position of the anatomical feature. Additionally, or alternatively, the system is configured to determine a size of the anatomical feature of the eye (e.g., based on the information on the outline of the anatomical feature), and to control the illumination system to adjust a width of the light beam based on the size of the anatomical feature of the eye, e.g., such that the light beam fits into the outline and/or fills the outline of the anatomical feature (as far as possible). In particular, this may be applied to the pupil, i.e., the anatomical feature may be the pupil. Accordingly, the system may be configured to control the illumination system to adjust the property of the light beam based on the information on the pupil of the eye. Alternatively, or additionally, the position and/or outline of the limbus may be used to determine the position of the pupil. Thus, the system may be configured to control the illumination system to adjust the property of the light beam based on the information on the limbus of the eye.

In FIGS. 1c to 1e, three different implementations are shown that allow adjusting the path and/or the width of the light beam. FIGS. 1c to 1e shows schematic diagrams of examples of an ophthalmic microscope system, and in particular of different illumination systems of the ophthalmic microscope system.

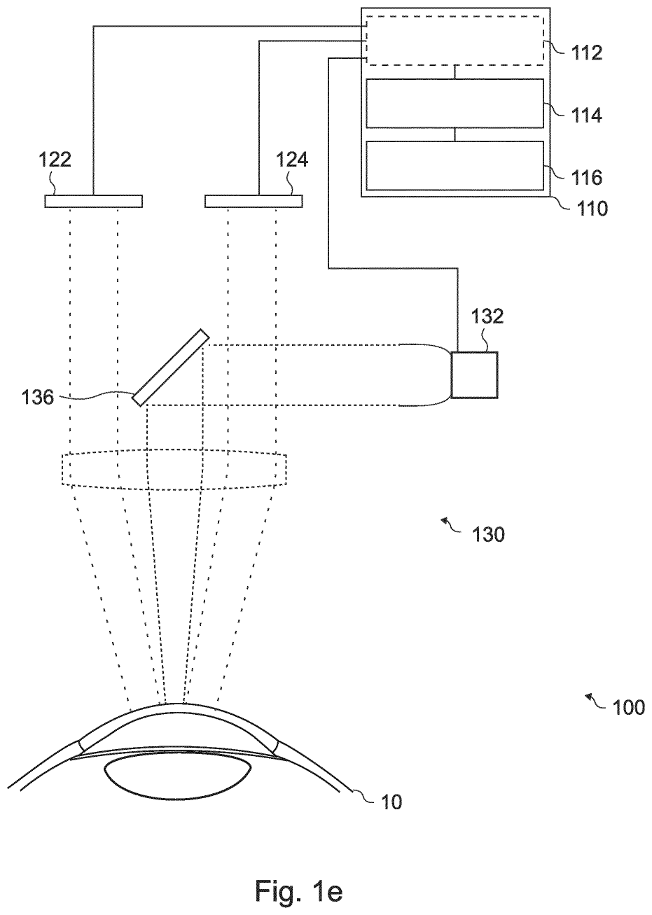

FIG. 1c shows an example of an ophthalmic microscope system 100 comprising the system 110, two optical imaging sensors 122; 124 (in a stereoscopic configuration), and the illumination system 130 with a light source 132 and an adjustable iris 134. For example, the illumination system 130 may further comprise one or more motors (not shown) for adjusting the position of at least one of the light source 132 and the iris 134 of the illumination system. For example, as shown in FIG. 4, the illumination system may comprise a platform 410 that hosts the iris 134, and which is movable in x-y-direction perpendicular to the light beam. Optionally, the light source may be moved together with the iris. Alternatively, a setup without an adjustable iris may be used, with the light source being movable in x-y-direction via a similar platform and corresponding motor or motors. The system may be configured to control the one or more motors to adjust the position of at least one of the light source and the iris of the illumination system in order to adjust a path of a light beam being emitted by the illumination system. In particular, the system may be configured to control the one or more motors to move the light source and/or the iris in x-y-direction perpendicular to the light beam (with the light beam defining the z-direction). By moving the light source and/or the iris, the light beam is shifted (i.e., displaced, offset) relative to the eye, to a position that intersects with the position of the pupil.

In addition to the path of the light beam, the setup of FIG. 1c may be used to adjust the width of the light beam (via the iris 134). For example, the system may be configured to control the iris 134 of the illumination system in order to adjust the width (or diameter) of the light beam. For example, as shown in FIG. 1c, the light beam may become narrower towards the eye (due to the optics of the objective). The iris 134 may be controlled such, that the width of the light beam upon intersection with the eye corresponds to the above-mentioned desired width of the light beam.

In FIGS. 1d and 1e, a different concept is used. In FIGS. 1d and 1e, a display device is used to modulate the light beam (i.e., to alter the properties of the light beam, such as path and width). FIGS. 1d and 1e show examples of an ophthalmic microscope system 100 comprising the system 110, two optical imaging sensors 122; 124 (in a stereoscopic configuration), and the illumination system 130 with a light source 132 and a display device 136 being configured to modulate the light beam. In other words, the illumination system 130 may comprise a display device 136 for modulating a light beam being emitted by the illumination system. The system may be configured to control the display device in order to adjust at least one of a path and a width of the light beam, as will be shown in the following.

FIGS. 1d and 1e shows two different examples, which differ primarily in the placement of the display device, and thus the concept being used to modulate the light beam. In FIG. 1d, the light beam is transmitted through the (at least partially transparent) display device. In this case, the display device 136 is configured to modulate the light beam as the light beam passes through the display device towards the eye. For example, in this configuration, the display device may be a liquid crystal display (LCD) without a backlight. In an LCD, two polarizing filter layers (having polarizations that are perpendicular to each other) are combined with a layer of liquid crystals, which, if turned “on” via one or more transistors, can modify the polarization of the light as it passes through the layer of liquid crystals. In an “off-state”, the polarization of the polarizing filters being perpendicular to each other blocks (or at least attenuates) light from passing through the LCD. If a liquid crystal of a pixel of the LCD is set to an “on-state” (which may vary in intensity), the polarization of the light passing through said pixel is changed, allowing the light to pass through the LCD. In effect, the LCD allows the definition of pixels that block (or at least severely attenuate) the light, and pixels that pass the light. The system may be configured to control the (LCD) display device to set a first set of pixels to block the light and a second set of pixels to pass the light, such that the path of the light beam and/or the width of the light beam is modulated (i.e., changed) to the desired path and/or width of the light beam (by setting a suitable first set of pixels to an “on-state”, such that the light beam passes through the first set of pixels.

In FIG. 1e, on the other hand, the light beam is reflected off the display device. In other words, the display device 136 may be configured to selectively reflect the light beam towards the eye, thereby modulating the light beam. In this case, similar to the configuration shown in FIG. 1d, a first set of pixels of the display device are set to reflect the light beam, and a second set of pixels of the display device are set to absorb the light (or at least reflect with less intensity). This may be achieved by setting the first set of pixels to white and the second set of pixels to black (if the display device is an LCD with backlight or a self-emitting display panel, such as an organic light emitting diode (OLED) display panel), or by setting the first set of pixels to reflect the light towards the eye and the second set of pixels to reflect the light somewhere else or to absorb the light (if the display device is a micro mirror array panel).

As outlined above, in some examples, the intensity of the light beam may be modulated, e.g., such that the red reflex is visible at a desired brightness or contrast. The system may be configured to determine a brightness or contrast of the red reflex illumination of the eye (e.g., the information on the intensity of the red reflex observed through the anatomical feature), e.g., from the imaging sensor data, and to control the illumination system to adjust an intensity of the light beam based on the brightness or contrast of the red reflex illumination. In particular, the system may be configured to adjust the intensity of the light beam such that the brightness or contrast of the red reflex matches a desired brightness or contrast, e.g., by iteratively increasing or decreasing the intensity of the light beam. Alternatively, a lookup table may be used to derive the intensity of the light beam from a perceived brightness or contrast of the red reflex. To adjust the intensity of the light beam, the system may control the light source to adjust the intensity of the light emitted by the light source, or to control the display device to adjust the intensity of the reflection and/or attenuation caused by the display device. In other words, the system may be configured to control the light source 132 of the illumination system in order to adjust the intensity of the light beam. Alternatively, the system may be configured to control the display device 136 in order to adjust the intensity of the light beam.

In addition to the light source 132 shown in FIGS. 1c to 1e, the illumination system may further comprise a separate light source that is used as main light for illuminating the eye during surgery. For example, the separate light source may be adjustable separately (by the system) from the light source 132 being used for red reflex illumination.

In general, the proposed concept may be used during surgery, to adjust the light beam to changes that occur during surgery. In other words, the proposed system may react to changes in the anatomical feature and adjust the light beam accordingly after a change (e.g., in response to a change). Accordingly, the system may be configured to update the information on the anatomical feature over a sequence of frames of the imaging sensor data, and to control the illumination system to adjust the property of the light beam upon detection of change in the information on the anatomical feature of the eye. For example, the system may be configured re-determine the information on the anatomical feature based on at least a subset of the frames of the imaging sensor data, e.g., based on every n-th frame (with n>1). If the information on the anatomical feature changes between frames being processed, the property of the light beam may be adjusted accordingly, e.g., if the changes between the two sets of information on the anatomical surpass a pre-defined threshold, e.g., a predefined distance threshold (for a distance between two positions or outlines) or a predefined brightness difference threshold (for a difference between two brightness levels).

In the proposed ophthalmic microscope system, at least one optical imaging sensor is used to provide the imaging sensor data. Accordingly, the at least one optical imaging sensor is configured to generate the imaging sensor data. For example, the at least one optical imaging sensor 122; 124 of the microscope 120 may comprise or be APS (Active Pixel Sensor)—or a CCD (Charge-Coupled-Device)-based imaging sensor. For example, in APS-based imaging sensors, light is recorded at each pixel using a photodetector and an active amplifier of the pixel. APS-based imaging sensors are often based on CMOS (Complementary Metal-Oxide-Semiconductor) or S-CMOS (Scientific CMOS) technology. In CCD-based imaging sensors, incoming photons are converted into electron charges at a semiconductor-oxide interface, which are subsequently moved between capacitive bins in the imaging sensors by a circuitry of the imaging sensors to perform the imaging. The system 110 may be configured to obtain (i.e., receive or read out) the imaging sensor data from the at least one optical imaging sensor. The imaging sensor data may be obtained by receiving the imaging sensor data from the at least one optical imaging sensor (e.g., via the interface 112), by reading the imaging sensor data out from a memory of the at least one optical imaging sensor (e.g., via the interface 112), or by reading the imaging sensor data from a storage device 116 of the system 110, e.g., after the imaging sensor data has been written to the storage device 116 by the at least one optical imaging sensor or by another system or processor.

The one or more interfaces 112 of the system 110 may correspond to one or more inputs and/or outputs for receiving and/or transmitting information, which may be in digital (bit) values according to a specified code, within a module, between modules or between modules of different entities. For example, the one or more interfaces 112 may comprise interface circuitry configured to receive and/or transmit information. The one or more processors 114 of the system 110 may be implemented using one or more processing units, one or more processing devices, any means for processing, such as a processor, a computer or a programmable hardware component being operable with accordingly adapted software. In other words, the described function of the one or more processors 114 may as well be implemented in software, which is then executed on one or more programmable hardware components. Such hardware components may comprise a general-purpose processor, a Digital Signal Processor (DSP), a micro-controller, etc. The one or more storage devices 116 of the system 110 may comprise at least one element of the group of a computer readable storage medium, such as a magnetic or optical storage medium, e.g., a hard disk drive, a flash memory, Floppy-Disk, Random Access Memory (RAM), Programmable Read Only Memory (PROM), Erasable Programmable Read Only Memory (EPROM), an Electronically Erasable Programmable Read Only Memory (EEPROM), or a network storage.

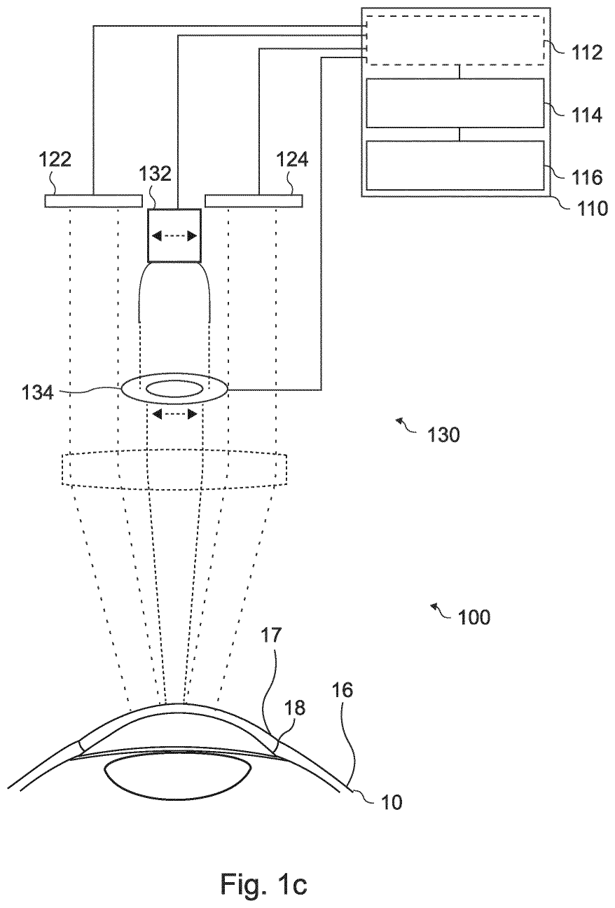

More details and aspects of the system and of the ophthalmic microscope system are mentioned in connection with the proposed concept or one or more examples described above or below (e.g., FIGS. 2 to 6). The system and/or the ophthalmic microscope system may comprise one or more additional optional features corresponding to one or more aspects of the proposed concept, or one or more examples described above or below.

FIG. 2 shows a flow chart of an example of a corresponding (computer-implemented) method for an ophthalmic microscope system. The method comprises obtaining 210 imaging sensor data from an optical imaging sensor of a microscope of the ophthalmic microscope system. The method comprises determining 220 information on an anatomical feature of an eye shown in the imaging sensor data. The method comprises controlling 230 an illumination system of the ophthalmic microscope system to adjust a property of a light beam used for red reflex illumination of the eye based on the information on the anatomical feature.

For example, the method may be performed by the system 110 and/or ophthalmic microscope system 100 introduced in connection with FIGS. 1a to 1e. Features introduced in connection with the system 110 and/or ophthalmic microscope system 100 may likewise be introduced in the corresponding method of FIG. 2.

More details and aspects of the method are mentioned in connection with the proposed concept, or one or more examples described above or below (e.g., FIG. 1a to 1e, 3 to 6). The method may comprise one or more additional optional features corresponding to one or more aspects of the proposed concept, or one or more examples described above or below.

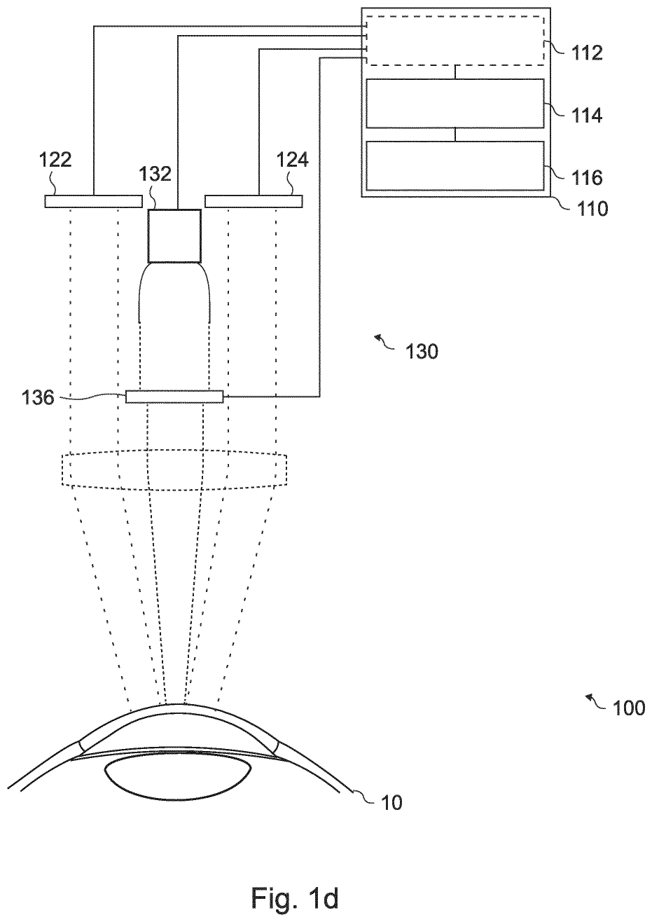

FIG. 3 shows a block diagram of an example of components of the proposed ophthalmic microscope system. In the example, the ophthalmic microscope system comprises a video camera 310 (e.g., the at least one optical imaging sensor 122;124 of the microscope 120 shown in FIGS. 1a to 1e), a processor 320 that is coupled to the video camera 310, a controller 330 that is coupled to the processor 320 (with the processor 320 and controller 310 potentially implemented by the system 110), and actuators 340 (e.g., the adjustable iris, the display device or the motors being used to move the iris, the light source) which are coupled to the controller 330.

The one or more video cameras 310 on the microscope may capture video of the surgical field and the video signals may then be fed to the processor 320. The processor 320 may analyze the microscope video content to detect the presence of an eye and localize it in the video frames. The diameter of limbus or pupil (i.e., the anatomical feature) may be automatically measured, and the corresponding red reflex light iris diameter may be selected. The processor may then instruct the controller 330 to move and resize the iris of the red reflex light by controlling the actuators 340.

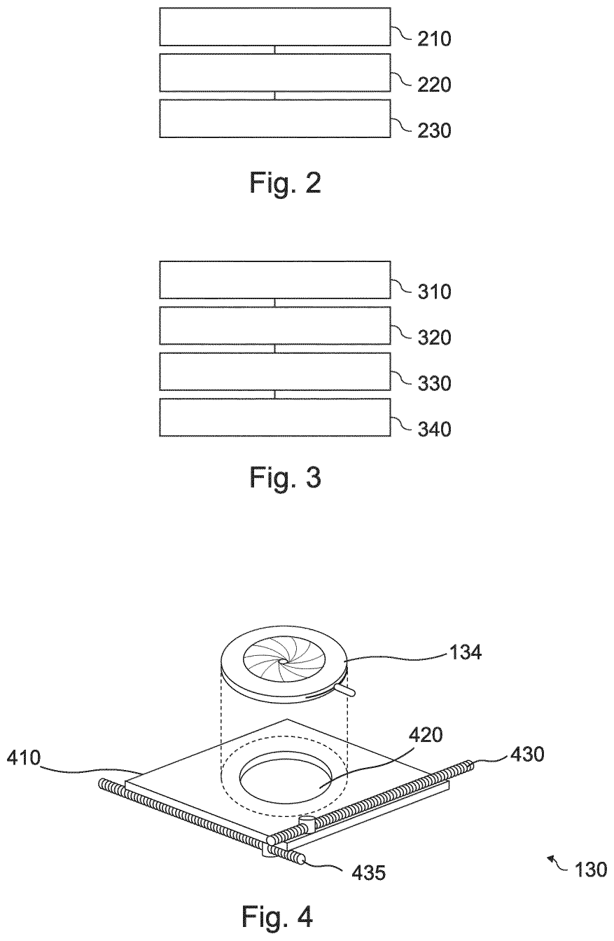

The iris can be an opto-mechanical apparatus mounted on a platform that can shift in a plane perpendicular to the illuminating optical path, as shown in FIG. 4. Alternatively, an electronic modulator with pixels such as a liquid-crystal display, a micro mirror array panel, or a self-emitting display panel (e.g., the display device 136) may be used, as shown in FIG. 5. In such cases, the illumination pattern may remain circular but might not have to be.

While detecting and locating the limbus and/or pupil, the processor may also measure the brightness of red reflex and instruct the red reflex light controller to change the illumination intensity (below a preset safety threshold) that offers clear visualization especially in digital video. For example, during hydrodissection, the light intensity may be increased to maintain clarity of red reflex.

FIG. 4 shows a schematic diagram of an example of a mechanically adjustable iris that may be part of the illumination system 130. The mechanically adjustable iris comprises the actual iris 134, which is an adjustable iris diaphragm, which is affixed to an x-y-movable platform 410, which comprises an opening 420 that is aligned with the iris 134 or that houses the iris 134. Shafts 430; 435 may be used for moving the platform 410 in x- and y-direction.

FIG. 5 shows a schematic diagram of an example of an electronically adjustable iris that may be part of the illumination system 130. Light 510 from the light source is emitted towards a reflective display device 136, with a portion 530 of the light that is incident to a portion 520 of the reflective display device 136 being reflected towards the projection lens 540.

More details and aspects of the ophthalmic microscope system are mentioned in connection with the proposed concept, or one or more examples described above or below (e.g., FIG. 1a to 2, 6). The ophthalmic microscope system may comprise one or more additional optional features corresponding to one or more aspects of the proposed concept, or one or more examples described above or below.

As used herein the term “and/or” includes any and all combinations of one or more of the associated listed items and may be abbreviated as “/”.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus.

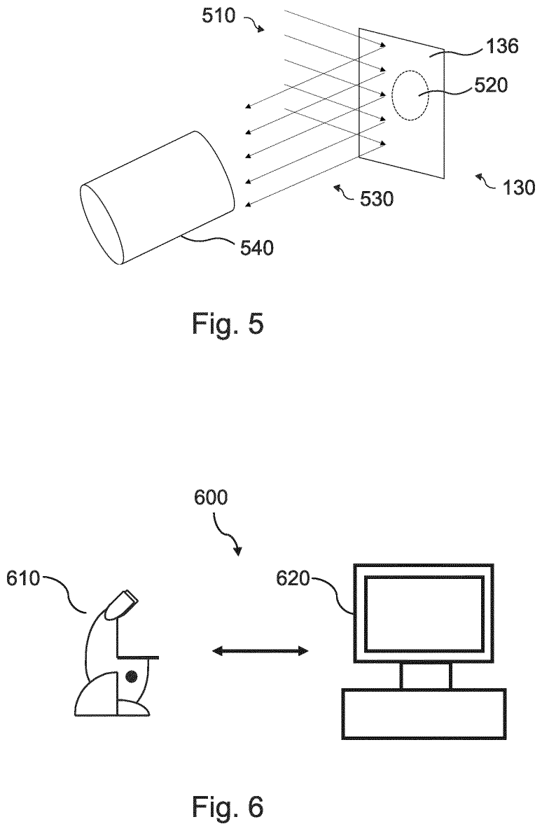

Some embodiments relate to a microscope comprising a system as described in connection with one or more of the FIGS. 1 to 5. Alternatively, a microscope may be part of or connected to a system as described in connection with one or more of the FIGS. 1 to 5. FIG. 6 shows a schematic illustration of a system 600 configured to perform a method described herein. The system 600 comprises a microscope 610 and a computer system 620. The microscope 610 is configured to take images and is connected to the computer system 620. The computer system 620 is configured to execute at least a part of a method described herein. The computer system 620 may be configured to execute a machine learning algorithm. The computer system 620 and microscope 610 may be separate entities but can also be integrated together in one common housing. The computer system 620 may be part of a central processing system of the microscope 610 and/or the computer system 620 may be part of a subcomponent of the microscope 610, such as a sensor, an actor, a camera or an illumination unit, etc. of the microscope 610.

The computer system 620 may be a local computer device (e.g. personal computer, laptop, tablet computer or mobile phone) with one or more processors and one or more storage devices or may be a distributed computer system (e.g. a cloud computing system with one or more processors and one or more storage devices distributed at various locations, for example, at a local client and/or one or more remote server farms and/or data centers). The computer system 620 may comprise any circuit or combination of circuits. In one embodiment, the computer system 620 may include one or more processors which can be of any type. As used herein, processor may mean any type of computational circuit, such as but not limited to a microprocessor, a microcontroller, a complex instruction set computing (CISC) microprocessor, a reduced instruction set computing (RISC) microprocessor, a very long instruction word (VLIW) microprocessor, a graphics processor, a digital signal processor (DSP), multiple core processor, a field programmable gate array (FPGA), for example, of a microscope or a microscope component (e.g. camera) or any other type of processor or processing circuit. Other types of circuits that may be included in the computer system 620 may be a custom circuit, an application-specific integrated circuit (ASIC), or the like, such as, for example, one or more circuits (such as a communication circuit) for use in wireless devices like mobile telephones, tablet computers, laptop computers, two-way radios, and similar electronic systems. The computer system 620 may include one or more storage devices, which may include one or more memory elements suitable to the particular application, such as a main memory in the form of random access memory (RAM), one or more hard drives, and/or one or more drives that handle removable media such as compact disks (CD), flash memory cards, digital video disk (DVD), and the like. The computer system 620 may also include a display device, one or more speakers, and a keyboard and/or controller, which can include a mouse, trackball, touch screen, voice-recognition device, or any other device that permits a system user to input information into and receive information from the computer system 620.

Some or all of the method steps may be executed by (or using) a hardware apparatus, like for example, a processor, a microprocessor, a programmable computer or an electronic circuit. In some embodiments, some one or more of the most important method steps may be executed by such an apparatus.

Depending on certain implementation requirements, embodiments of the invention can be implemented in hardware or in software. The implementation can be performed using a nontransitory storage medium such as a digital storage medium, for example a floppy disc, a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. Therefore, the digital storage medium may be computer readable.

Some embodiments according to the invention comprise a data carrier having electronically readable control signals, which are capable of cooperating with a programmable computer system, such that one of the methods described herein is performed.

Generally, embodiments of the present invention can be implemented as a computer program product with a program code, the program code being operative for performing one of the methods when the computer program product runs on a computer. The program code may, for example, be stored on a machine readable carrier.

Other embodiments comprise the computer program for performing one of the methods described herein, stored on a machine readable carrier.

In other words, an embodiment of the present invention is, therefore, a computer program having a program code for performing one of the methods described herein, when the computer program runs on a computer.

A further embodiment of the present invention is, therefore, a storage medium (or a data carrier, or a computer-readable medium) comprising, stored thereon, the computer program for performing one of the methods described herein when it is performed by a processor. The data carrier, the digital storage medium or the recorded medium are typically tangible and/or non-transitionary. A further embodiment of the present invention is an apparatus as described herein comprising a processor and the storage medium.

A further embodiment of the invention is, therefore, a data stream or a sequence of signals representing the computer program for performing one of the methods described herein. The data stream or the sequence of signals may, for example, be configured to be transferred via a data communication connection, for example, via the internet.

A further embodiment comprises a processing means, for example, a computer or a programmable logic device, configured to, or adapted to, perform one of the methods described herein.

A further embodiment comprises a computer having installed thereon the computer program for performing one of the methods described herein.

A further embodiment according to the invention comprises an apparatus or a system configured to transfer (for example, electronically or optically) a computer program for performing one of the methods described herein to a receiver. The receiver may, for example, be a computer, a mobile device, a memory device or the like. The apparatus or system may, for example, comprise a file server for transferring the computer program to the receiver.

In some embodiments, a programmable logic device (for example, a field programmable gate array) may be used to perform some or all of the functionalities of the methods described herein. In some embodiments, a field programmable gate array may cooperate with a microprocessor in order to perform one of the methods described herein. Generally, the methods are preferably performed by any hardware apparatus.

Some examples of the present disclosure are based on using a machine-learning model or machine-learning algorithm.

Machine-learning models may be trained using training input data. The examples specified above use a training method called “supervised learning”. In supervised learning, the machine-learning model is trained using a plurality of training samples, wherein each sample may comprise a plurality of input data values, and a plurality of desired output values, i.e. each training sample is associated with a desired output value. By specifying both training samples and desired output values, the machine-learning model “learns” which output value to provide based on an input sample that is similar to the samples provided during the training. Apart from supervised learning, semi-supervised learning may be used. In semi-supervised learning, some of the training samples lack a corresponding desired output value. Supervised learning may be based on a supervised learning algorithm (e.g. a classification algorithm, a regression algorithm or a similarity learning algorithm. Classification algorithms may be used when the outputs are restricted to a limited set of values (categorical variables), i.e. the input is classified to one of the limited set of values. Regression algorithms may be used when the outputs may have any numerical value (within a range). Similarity learning algorithms may be similar to both classification and regression algorithms but are based on learning from examples using a similarity function that measures how similar or related two objects are.

Apart from supervised or semi-supervised learning, unsupervised learning may be used to train the machine-learning model. In unsupervised learning, (only) input data might be supplied and an unsupervised learning algorithm may be used to find structure in the input data (e.g. by grouping or clustering the input data, finding commonalities in the data). Clustering is the assignment of input data comprising a plurality of input values into subsets (clusters) so that input values within the same cluster are similar according to one or more (pre-defined) similarity criteria, while being dissimilar to input values that are included in other clusters.

Reinforcement learning is a third group of machine-learning algorithms. In other words, reinforcement learning may be used to train the machine-learning model. In reinforcement learning, one or more software actors (called “software agents”) are trained to take actions in an environment. Based on the taken actions, a reward is calculated. Reinforcement learning is based on training the one or more software agents to choose the actions such, that the cumulative reward is increased, leading to software agents that become better at the task they are given (as evidenced by increasing rewards).

Furthermore, some techniques may be applied to some of the machine-learning algorithms. For example, feature learning may be used. In other words, the machine-learning model may at least partially be trained using feature learning, and/or the machine-learning algorithm may comprise a feature learning component. Feature learning algorithms, which may be called representation learning algorithms, may preserve the information in their input but also transform it in a way that makes it useful, often as a pre-processing step before performing classification or predictions. Feature learning may be based on principal components analysis or cluster analysis, for example.

In some examples, anomaly detection (i.e. outlier detection) may be used, which is aimed at providing an identification of input values that raise suspicions by differing significantly from the majority of input or training data. In other words, the machine-learning model may at least partially be trained using anomaly detection, and/or the machine-learning algorithm may comprise an anomaly detection component.

In some examples, the machine-learning algorithm may use a decision tree as a predictive model. In other words, the machine-learning model may be based on a decision tree. In a decision tree, observations about an item (e.g. a set of input values) may be represented by the branches of the decision tree, and an output value corresponding to the item may be represented by the leaves of the decision tree. Decision trees may support both discrete values and continuous values as output values. If discrete values are used, the decision tree may be denoted a classification tree, if continuous values are used, the decision tree may be denoted a regression tree.

Association rules are a further technique that may be used in machine-learning algorithms. In other words, the machine-learning model may be based on one or more association rules. Association rules are created by identifying relationships between variables in large amounts of data. The machine-learning algorithm may identify and/or utilize one or more relational rules that represent the knowledge that is derived from the data. The rules may e.g. be used to store, manipulate or apply the knowledge.

Machine-learning algorithms are usually based on a machine-learning model. In other words, the term “machine-learning algorithm” may denote a set of instructions that may be used to create, train or use a machine-learning model. The term “machine-learning model” may denote a data structure and/or set of rules that represents the learned knowledge (e.g. based on the training performed by the machine-learning algorithm). In embodiments, the usage of a machine-learning algorithm may imply the usage of an underlying machine-learning model (or of a plurality of underlying machine-learning models). The usage of a machine-learning model may imply that the machine-learning model and/or the data structure/set of rules that is the machine-learning model is trained by a machine-learning algorithm.

For example, the machine-learning model may be an artificial neural network (ANN). ANNs are systems that are inspired by biological neural networks, such as can be found in a retina or a brain. ANNs comprise a plurality of interconnected nodes and a plurality of connections, so-called edges, between the nodes. There are usually three types of nodes, input nodes that receiving input values, hidden nodes that are (only) connected to other nodes, and output nodes that provide output values. Each node may represent an artificial neuron. Each edge may transmit information, from one node to another. The output of a node may be defined as a (non-linear) function of its inputs (e.g. of the sum of its inputs). The inputs of a node may be used in the function based on a “weight” of the edge or of the node that provides the input. The weight of nodes and/or of edges may be adjusted in the learning process. In other words, the training of an artificial neural network may comprise adjusting the weights of the nodes and/or edges of the artificial neural network, i.e. to achieve a desired output for a given input.

Alternatively, the machine-learning model may be a support vector machine, a random forest model or a gradient boosting model. Support vector machines (i.e. support vector networks) are supervised learning models with associated learning algorithms that may be used to analyze data (e.g. in classification or regression analysis). Support vector machines may be trained by providing an input with a plurality of training input values that belong to one of two categories. The support vector machine may be trained to assign a new input value to one of the two categories. Alternatively, the machine-learning model may be a Bayesian network, which is a probabilistic directed acyclic graphical model. A Bayesian network may represent a set of random variables and their conditional dependencies using a directed acyclic graph. Alternatively, the machine-learning model may be based on a genetic algorithm, which is a search algorithm and heuristic technique that mimics the process of natural selection.

### LIST OF REFERENCE SIGNS

10 Eye
12 Pupil
14 Iris
16 Sclera
17 Cornea
18 Limbus
100 Ophthalmic microscope system
105 Stand
110 System
112 One or more interfaces
114 One or more processors
116 One or more storage devices
120 Microscope
122; 124 Optical imaging sensor
130 Illumination system
132 Light source
134 Iris
136 Display device
140a Ocular displays
140b Auxiliary display
150 Arm
210 Obtaining imaging sensor data
220 Determining information on an anatomical feature
230 Controlling an illumination system
310 Video camera
320 Processor
330 Controller
340 Actuators
410 Platform
420 Opening
430; 435 Shaft
510 Light from light source
520 Portion of reflective display device
530 Light reflected towards a projection lens
540 Projection lens
600 System
610 Microscope
620 Computer system

1. A system for an ophthalmic microscope system, the system comprising one or more processors and one or more storage devices, wherein the system is configured to:
obtain imaging sensor data from an optical imaging sensor of a microscope of the ophthalmic microscope system;
determine information on an anatomical feature of an eye shown in the imaging sensor data; and
control an illumination system of the ophthalmic microscope system to adjust a property of a light beam used for red reflex illumination of the eye based on the information on the anatomical feature.

2. The system according to claim 1, wherein the anatomical feature is a pupil of the eye, wherein the system is configured to determine information on the pupil of the eye, and to control the illumination system to adjust the property of the light beam based on the information on the pupil of the eye.

3. The system according to claim 1, wherein the anatomical feature is a limbus of the eye, wherein the system is configured to determine information on the limbus of the eye, and to control the illumination system to adjust the property of the light beam based on the information on the limbus of the eye.

4. The system according to claim 1, wherein the system is configured to determine a position of the anatomical feature of the eye, and to control the illumination system to adjust a path of the light beam based on the position of the anatomical feature of the eye.

5. The system according to claim 1, wherein the system is configured to determine a size of the anatomical feature of the eye, and to control the illumination system to adjust a width of the light beam based on the size of the anatomical feature of the eye.

6. The system according to claim 1, wherein the system is configured to determine a brightness or contrast of the red reflex illumination of the eye, and to control the illumination system to adjust an intensity of the light beam based on the brightness or contrast of the red reflex illumination.

7. The system according to claim 1, wherein the system is configured to update the information on the anatomical feature over a sequence of frames of the imaging sensor data, and to control the illumination system to adjust the property of the light beam upon detection of change in the information on the anatomical feature of the eye.

8. The system according to claim 1, wherein the system is configured to detect the anatomical feature within the imaging sensor data using a machine-learning model being trained to detect the anatomical feature in imaging sensor data, and to determine the information on the anatomical feature based on an output of the machine-learning model.

9. The system according to claim 8, wherein the output of the machine-learning model comprises at least one of information on a position and information on an outline of the anatomical feature.

10. The system according to claim 1, wherein the system is configured to control the illumination system to temporarily increase a beam width of the light beam, and to determine the information on the anatomical feature based on imaging sensor data generated based on the increased beam width.

11. The system according to claim 1, wherein the system is configured to control the illumination system to temporarily disable the light beam, to compare imaging sensor data generated while the light beam is disabled with imaging sensor data generated while the light beam is enabled, and to determine the information on the anatomical feature based on the comparison.

12. An ophthalmic microscope system, comprising the system according to claim 1, the microscope and the illumination system.

13. The ophthalmic microscope system according to claim 12, wherein the illumination system comprises one or more motors for adjusting the position of at least one of a light source and an iris of the illumination system, and wherein the system is configured to control the one or more motors to adjust the position of at least one of a light source and an iris of the illumination system in order to adjust a path of a light beam being emitted by the illumination system.

14. The ophthalmic microscope system according to claim 12, wherein the illumination system comprises a display device for modulating a light beam being emitted by the illumination system, wherein the system is configured to control the display device in order to adjust at least one of a path and a width of the light beam.

15. The ophthalmic microscope system according to claim 14, wherein the illumination system comprises a light source being configured to emit the light beam and the display device, wherein the display device is configured to selectively reflect the light beam towards the eye, thereby modulating the light beam.

16. The ophthalmic microscope system according to claim 14, wherein the illumination system comprises a light source being configured to emit the light beam and the display device, wherein the display device is configured to modulate the light beam as the light beam passes through the display device towards the eye.

17. A method for an ophthalmic microscope system, the method comprising:
obtaining imaging sensor data from an optical imaging sensor of a microscope of the ophthalmic microscope system;
determining information on an anatomical feature of an eye shown in the imaging sensor data; and
controlling an illumination system of the ophthalmic microscope system to adjust a property of a light beam used for red reflex illumination of the eye based on the information on the anatomical feature.

18. A non-transitory, computer-readable medium comprising a program code that when the program code is executed on a processor, a computer, or a programmable hardware component, causes the processor, computer, or programmable hardware component to perform the method of claim 17.
